(19) United States
(12) Patent Application Publication
(10) Pub. No.: US 2012/0309098 A1
(43) Pub. Date: Dec. 6, 2012

(54) METHOD FOR PREDICTING THE AMOUNTS OF BIOGENIC GAS GENERATED BY BIOLOGICAL CONVERSION OF ORGANIC MATTER

(76) Inventors: BEHAR, (US); Haeseler, (US)

(21) Appl. No.: 13/463,938

(22) Filed: May 4, 2012

(30) Foreign Application Priority Data
Jun. 5, 2011 (FR) 11/01.402

(51) Int. Cl. G01N 33/24

(52) U.S. Cl. 436/141

(57) ABSTRACT

Method for predicting amounts of biogenic gas generated by biological conversion of organic matter deposited in sediments on the geological time scale.

Bioreactive structural units having formulae CₓH_{y}OᵥS_{z}N_{w} with x>6, y>x, v<6, z<6 and w<6 are considered. A sample of a rock of the sedimentary series is then taken at a depth close to the surface, such that said rock has not been subjected to any biodegradation, and amounts of each bioreactive structural unit present in this sample are determined. A reaction scheme is defined in which the bioreactive structural units react in parallel with electron acceptors, and each of the bioreactive structural units reacts sequentially with these electron acceptors. Finally, the amount of biogenic gas produced by biodegradation of the bioreactive structural units is determined by means of the reaction scheme and the amounts of each structural unit present in the sample.

### The present invention relates to the field of the oil industry and more particularly to the field of exploration for and the production of fossil energy resulting from the subsoil.

The invention relates in particular to a method for evaluating the volumes of biogenic gas generated in sedimentary basins. It thus provides a very useful evaluation tool, in particular for geologists, for directing the investigations of new sources of fossil energy, such as methane present in relatively shallow sediments (between 0 and 2500 meters in depth).

One of the strategic challenges in oil exploration is to postpone as far as possible the exhausting of hydrocarbon resources. In this context, zones never explored due to the production costs are now regarded as potentially exploitable. Among these new resources, biogenic gas accumulated in a relatively shallow sediments (less than 2500 m) is currently regarded as a potential new reserve of fossil energy.

Unlike the phenomena of biodegradation of oils accumulated in reservoirs, which detrimentally affect their quality by destroying the lightest hydrocarbons, microbial activity in sediments consumes a portion of the carbon present in the sediments to covert it into biogenic gas, which in particular is a mixture of CO₂ and methane under the conditions of methanogenesis. As it is possible for the CO₂ to dissolve in water, the proportion of methane in the gas phase can then become predominant.

Biodegradation is a biological phenomenon which can affect the organic matter deposited in sediments. The bacteria responsible for these reactions are present in the porous surroundings and live in the water which circulates through the porous surroundings. These bacteria use the organic matter present as source of carbon and energy in order to provide for their growth and their maintenance. In doing this, on the one hand, they selectively decompose certain structures of this organic matter and thus modify both the amount and the composition of this organic matter in place and, on the other hand, they generate metabolites, such as gaseous entities (CH₄, H₂S and CO₂) and new chemical structures, such as carboxylic acids, which in their turn are decomposed. This source of carbon and energy is insufficient to ensure bacterial metabolism; it is also necessary for ingredients, such as electron acceptors and optionally nutrients, to be present in the system. The biological reactions do not need nutrients, such as phosphorus and nitrogen, and metals; however, these elements are essential for the synthesis of the constituent molecules of the bacteria. Their absence means that bacterial growth halts but their activity does not.

The biodegradation process can be described in the following way. The microorganisms present in the porous surroundings use the source of energy and carbon composed of certain chemical structures present in the organic matter with two objectives:
to grow and to produce as much biomass as possible, as long as the nitrogen and the phosphorus are not exhausted and as long as the trace elements, such as the metals, are not limiting factors,
to persist, that is to say to use the energy available in the organic matter through decomposition reactions which do not generate additional biomass. During these reactions, the sedimentary organic matter is effectively decomposed and metabolites are generated but no increase in biomass is observed.

In the oil industry, it is very important to know the role of the biological conversion of the sedimentary organic matter in order to know the amount of biogenic gas produced at shallow depth in a sedimentary basin. In order to achieve this, it is necessary to have available a method which makes it possible to estimate the effect of the biodegradation on the amount and quality of the starting organic matter and consequently on the volumetric analysis of the methane generated.

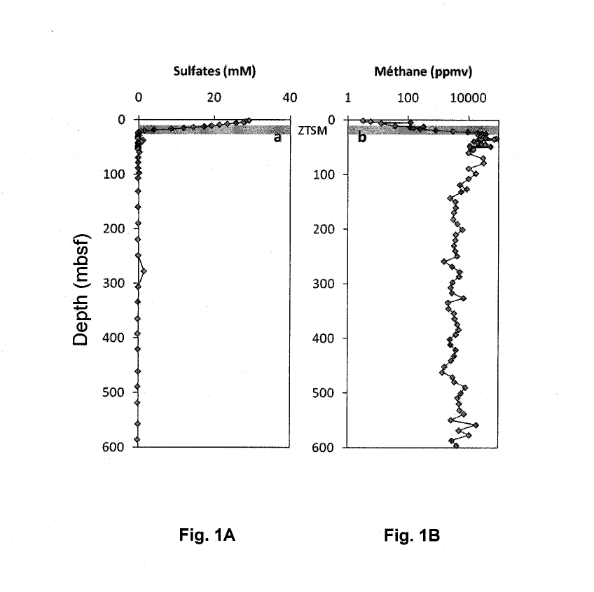

The microbial reactions for the conversion of sedimentary organic matter are successive reactions which begin by the decomposition in aerobic surroundings with consumption of oxygen (dissolved in the water), followed by the denitrification as soon as conditions become anoxic, with consumption of the nitrates (also dissolved in the water), followed by sulfate reduction under anaerobic conditions with consumption of the sulfates (which for their part can either be dissolved in the water or present in the form of precipitated salts in the rock). FIG. 1, in which the H₂S and CH₄ contents in the sedimentary series are plotted as a function of the depth, entirely illustrates these successive reactions.

The final reaction is methanogenesis under anaerobic conditions with consumption of the water present in the surroundings. The data available in the literature are mainly molecular characterizations of gases in sampling and coring campaigns in various regions of the globe. The organic matter, which is present virtually essentially in the solid form, is itself characterized as a function of the depth by overall methods, such as elemental assays for C, H, O, N and S. Nevertheless, the free organic carbon molecules associated with this organic matter can be extracted with appropriate solvents in order to determine the amount thereof and to carry out the molecular analysis thereof by mass spectrometry.

All these studies have formed the subject of numerous papers published in the literature. Nevertheless, the known methods are targeted at describing the biodegradation and not at predicting it. Specifically, there is always provided an overall equation for decomposition of the organic matter of the type:

organic matter==>CO₂+4H₂==>CH₄+2H₂O

This model does not take into account the structural change in the organic matter and accepts that all of the latter can be decomposed. In point of fact, it is well known that the effectiveness of the decomposition of this organic matter depends on the carbon-based structures present in the latter. Thus, it is expected that, depending on the chemical structure of the organic matter present, the rates and the amounts of biogenic gas generated will be variable. If such was not the case, organic carbon would not persist at depths greater than 2500 m and thus there would be no kerogen, the source of the formation of oil and gas.

Thus, the subject matter of the invention relates to a method for evaluating the amount of biogenic gas generated by biodegradation of organic matter in a sedimentary series of a sedimentary basin. The method is based on an analysis of the structural units of formulae CₓH_{y}OᵥS_{z}N_{w} with x>6, y>x, v<6, z<6 and w<6, and also on a the reaction scheme in which these structural units react in parallel with electron acceptors and sequentially with electron acceptors.

The method according to the invention provides a very useful evaluation tool, in particular for geologists preoccupied with directing investigations into new sources of fossil energy. In particular, it can be used in the technical field of basin modeling to predict the amount and the quality of the hydrocarbon gas which it may be expected to find in the relatively shallow sediments, which gas is related to a microbial alteration in the sedimentary organic matter under methanogenesis conditions.

### THE METHOD ACCORDING TO THE INVENTION

Generally, the invention relates to a method for determining an amount of biogenic gas generated by biodegradation of organic matter in a sedimentary series of a sedimentary basin. It comprises the following stages:
i. bioreactive structural units having formulae CₓH_{y}OᵥS_{z}N_{w} with x>6, y>x, v<6, z<6 and w<6 are considered;
ii. a sample of a rock of the sedimentary series is taken at a depth close to the surface, such that said rock has not been subjected to any biodegradation, and amounts of each bioreactive structural unit present in said sample are determined;
iii. a reaction scheme is defined in which said bioreactive structural units react in parallel with electron acceptors, and each of the bioreactive structural units reacts sequentially with said electron acceptors according to individual reaction rates for each bioreactive structural unit, said rates being determined by experimental measurements;
iv. the amount of biogenic gas produced by biodegradation of said bioreactive structural units is determined by means of said reaction scheme and said amounts of each structural unit present in said sample.

According to the invention, the individual reactions rates can be determined by carrying out the following stages:
a. rock samples are taken from a sedimentary series of a sedimentary basin, at the surface and at different depths;
b. a proportion by weight of each bioreactive structural unit is determined in each of said samples by means of extraction by an organic solvent, followed by pyrolysis;
c. an amount of biogenic gas produced by each biorective structural unit is determined by means of said reaction scheme and the proportions by weight;
d. the individual reaction rates of each bioreactive structural unit are deduced therefrom.

According to the invention, the bioreactive structural units can be grouped together into bioreactive structural groups. Finally, the biogenic gas can be methane and the electron acceptors can be chosen from the following acceptors: O₂, NO₃, SO₄ and H₂O.

Other characteristics and advantages of the method according to the invention will become apparent on reading the description below of nonlimiting implementational examples, reference being made to the appended figures described below.

FIG. 1 illustrates the change in the concentration of sulfates (a) and of methane (b) in interstitial water (according to Wefer et al., 1998); the level in gray represents the sulfate-methane transition zone (SMTZ).

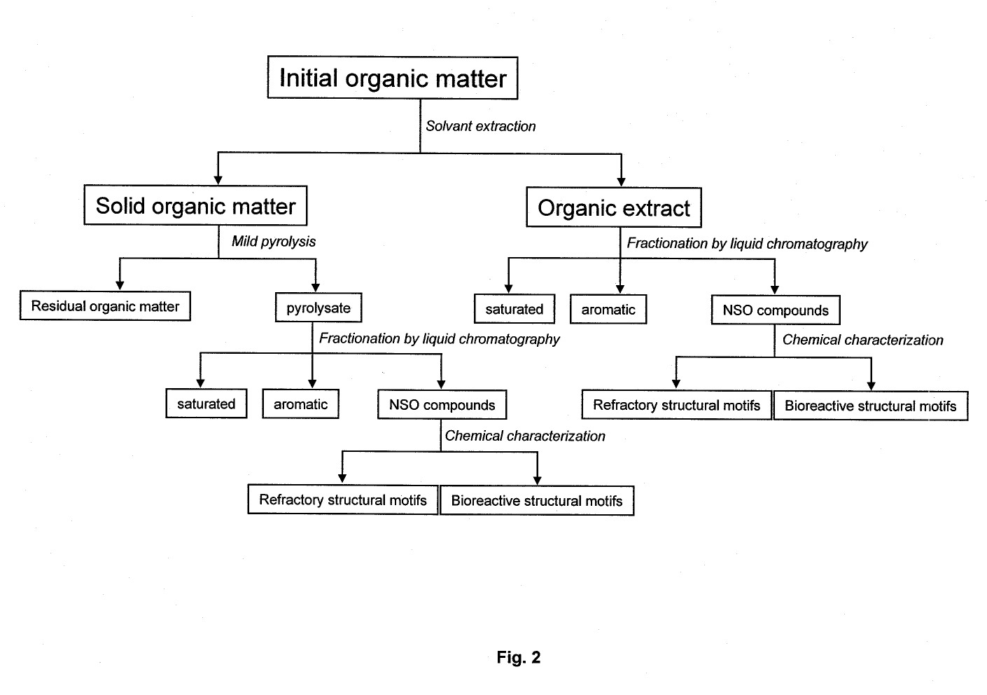

FIG. 2 illustrates the combined experimental approach in characterizing and quantifying the bioreactive and refractory structural units in the solid organic matter and the associated extract.

The method according to the invention makes it possible to estimate the amount of biogenic gas generated by biodegradation of organic matter in a rock of a sedimentary series during successive biological processes: aerobic, sulfate reduction and methanogenesis. For this, the change in the chemical structure of the organic matter is evaluated by carrying out a balance of bioreactive structural units which disappear during the biological processes and in particular during methanogenesis. This is because sedimentary organic matter is a complex macromolecule of very high molecular weight which comprises both biopolymers resulting from living organisms and also lipids having varied chemical structures connected to one another via functional groups.

The method mainly comprises the following stages:
1. the bioreactive structural units of the organic matter are defined;
2. a compositional estimation of the bioreactive structural units before biodegradation is carried out;
3. a reaction scheme for biodegradation of the bioreactive structural units is defined;
4. the amount of biogenic gas produced by biodegradation of the bioreactive structural units is evaluated by means of the reaction scheme and the amounts.

### 1—Bioreactive Structural Units

The microbial action which is the source of the biodegradation of sedimentary organic matter generates nonhydrocarbon gases (CO₂, H₂S) and methane (CH₄), referred to as “biogenic gas”. It sequentially alters the oxygen-based structures, in combination or not in combination with sulfur and nitrogen, present in the starting organic matter.

The term bioreactive structural units refers to the chemical structures of the initial organic matter which are capable of being biodegraded.

According to the invention, the structural units of formulae CₓH_{y}OᵥS_{z}N_{w} with x>6, y>x, v<6, z<6 and w<6 are considered in describing the group of the bioreactive structural units:

| Chemical structure of the bioreactive | Chemical functional groups | | | | | | | | | | | |
|---|---|---|---|---|---|---|---|---|---|---|---|---|
| structural units | acids | | | | alcohols | | | | ketones | | | |
| CₓH_{y}OᵥS_{z}N_{w}: with x > 6, y > x, v < 6, z < 6 and w < 6 | mono- | di- | tri- | poly- | mono- | di- | tri- | poly- | mono- | di- | tri- | poly- |
| linear | x | x | x | x | x | x | x | x | x | x | x | x |
| linear branched | x | x | x | x | x | x | x | x | x | x | x | x |
| saturated monocyclic | x | x | x | x | x | x | x | x | x | x | x | x |
| saturated dicyclic | x | x | x | x | x | x | x | x | x | x | x | x |
| saturated tricyclic | x | x | x | x | x | x | x | x | x | x | x | x |
| saturated tetracyclic | x | x | x | x | x | x | x | x | x | x | x | x |
| saturated pentacyclic | x | x | x | x | x | x | x | x | x | x | x | x |
| saturated polycyclic with N > 5 | x | x | x | x | x | x | | x | x | x | x | x |
| aromatic monocyclic | x | x | x | x | x | x | x | x | x | x | x | x |
| aromatic dicyclic | x | x | x | x | x | x | x | x | x | x | x | x |
| aromatic tricyclic | x | x | x | x | x | x | x | x | x | x | x | x |
| aromatic tetracyclic | x | x | x | x | x | x | x | x | x | x | x | x |
| aromatic pentacyclic | x | x | x | x | x | x | x | x | x | x | x | x |
| aromatic polycyclic with N > 5 | x | x | x | x | x | x | x | x | x | x | x | x |

All these bioreactive structural units constitute the bioreactive portion of the initial organic matter, referred to as “bioreactive organic matter”. These structures can be grouped together for calculation simplification issues.

### 2—Compositional Estimation of the Bioreactive Structural Units Before Biodegradation

In order to quantify the production of biogenic gas, it is necessary to determine and quantify the bioreactive and nonbioreactive structural units in the rock extracts and in the starting organic matter (before biodegradation). The assays of oxygen dissolved in water, those of H₂S and of methane as a function of the depth of the sediments make it possible to establish the depth ranges in which the biological reactions take place. In particular, the beginning of the zone of methanogenesis is defined by the virtually complete disappearance of H₂S. The organic matter taken from this zone is thus regarded as the initial organic matter before methanogenesis, which process is the source of the biogenic gas. To do this, the following stages are carried out:
a rock situated in a surface condition, that is to say at less than 10 m in depth, is isolated from a sedimentary series;
the inorganic matrix is destroyed in order to concentrate the organic matter, for example by using the method of Durand and Nicaise (1980);
the soluble organic compounds are extracted with organic solvents, such as, for example, dichloromethane. They constitute the rock extract;
the extract recovered is quantified by weighing;
the structural units are identified by appropriate methods and quantified;
a gentle pyrolysis is carried out, that is to say under anoxic conditions, on the extracted organic matter;
the structural units released during the gentle pyrolysis are recovered by extraction with organic solvents;
the structural units released during the pyrolysis are quantified by weighing;
the structural units released during the pyrolysis are characterized by appropriate methods;
a list of percentage of the various structural units present in the extract and in the pyrolysate is thus drawn up.

For the rock extracts, the determination of the bioreactive structural units in the saturated and aromatic hydrocarbons are analyzed, for example, by means of the 2D GC technique, and the structures comprising heteroelements to be analyzed, for example, by Fourier transform high resolution mass spectrometry or, for example, by conversion into hydrocarbon equivalents. For the solid organic matter, it is not possible to directly characterize the structural units thereof. According to the invention, the organic matter is heated under an inert atmosphere at a temperature of less than 225° C. for periods of time of 3 h to 24 h in order to have satisfactory yields. Varied organic molecules which are constituents of the organic matter are thus released. Saturated and aromatic hydrocarbons can be analyzed by known methods, for example 2D GC, and structures comprising heteroelements can be analyzed, for example, by Fourier transform high resolution mass spectrometry or, for example, by conversion into hydrocarbon equivalents. This pyrolysis releases the structural units carrying oxygen-based, sulfur-based and nitrogen-based functional groups without detrimentally affecting the latter. Thus, by carrying out the same pyrolyses and the same analyses on biodegraded organic matter, the amount and the quality of the structural units which have been destroyed during microbial alteration and which are thus bioreactive are determined by difference. It is also possible to determine, by this method, the amount and the quality of the structural units which would on the contrary be produced be this same alteration.

The whole of this experimental method is described in FIG. 2.

### 3—Compositional Scheme of the Bioreactive Structural Units

According to the invention, the biodegradation reaction scheme can comprise a group of sequential reactions for each bioreactive structural unit present in the starting organic matter (extract and solid organic matter) and which would have disappeared during the various alteration processes. The sequential equations for biodegradation of the bioreactive structural units with the main electron acceptors (O₂, NO₃, SO₄ and H₂O) are presented below.

### Electron Acceptor: O₂, Aerobic Biodegradation

$${C}_{x} {H}_{y} {O}_{z} {S}_{v} {N}_{w} + ( x + \frac{1}{4} y - \frac{1}{2} z + 2 v + \frac{2}{3} w ) {O}_{2} \to {x CO}_{2} + ( \frac{1}{2} y ) {H}_{2} O + {v SO}_{4} + {w NO}_{3} \qquad eq . 1$$

### Electron Acceptor: NO₃, Denitrification

$${C}_{x} {H}_{y} {O}_{z} {S}_{v} {N}_{w} + \frac{1}{3} ( 2 x + \frac{1}{2} y - z + 4 v ) {NO}_{3}^{-} \to {x CO}_{2} + \frac{1}{2} {y H}_{2} O + v {SO}_{4} + [ \frac{1}{6} ( 2 x + \frac{1}{2} y - y + 4 v ) + \frac{1}{2} w ] {N}_{2} \qquad eq . 2$$

### Electron Acceptor: SO₄, Sulfate Reduction

$${C}_{x} {H}_{y} {O}_{z} {S}_{v} {N}_{w} + \frac{1}{5} ( 2 x + \frac{1}{2} y - z - v ) {( S O )}_{4} \to {x CO}_{2} + \frac{1}{5} ( 2 y - 2 x + z - 4 v ) {H}_{2} O + \frac{1}{5} ( 2 x + \frac{1}{2} y - z + 4 v ) {H}_{2} S + \frac{1}{2} {w N}_{2} \qquad eq . 3$$

### Electron Acceptor: H₂O, Methanogenesis

$$\begin{matrix}{C}_{x} {H}_{y} {O}_{z} {S}_{v} {N}_{w} + ( x - z ) {H}_{2} O \to \frac{1}{2} {x CH}_{3} {CO}_{2}^{-} + \frac{1}{2} {x H}^{+} + \frac{1}{2} ( y - z - v ) {H}_{2} + {v H}_{2} S + \frac{1}{2} {w N}_{2} & eq . 4 \\ {CH}_{3} {CO}_{2}^{-} + {H}^{+} \to {CH}_{4} + {CO}_{2} & eq . 5 \\ {CO}_{2} + 4 {H}_{2} \to {CH}_{4} + 2 {H}_{2} O & eq . 6 \\ {C}_{x} {H}_{y} {O}_{z} {S}_{v} {N}_{w} + ( x - \frac{1}{4} y - \frac{1}{2} y + \frac{1}{2} v ) {H}_{2} O \to \frac{1}{2} ( x - \frac{1}{4} y + \frac{1}{2} z + \frac{1}{2} v ) {CO}_{2} + \frac{1}{2} ( x + \frac{1}{4} y - \frac{1}{2} z - \frac{1}{2} v ) {CH}_{4} + {v H}_{2} S + \frac{1}{2} {w N}_{2} & eq . 7\end{matrix}$$

Thus, the biodegradation reaction scheme according to the invention is defined by the group of the following chemical reactions:
for each bioreactive structural unit, the reactions with the electron acceptors (oxygen, nitrate, sulfate or H₂O) are sequential: each structural unit reacts with the electron acceptor having the highest oxidation/reduction potential, until this acceptor has been exhausted from the water, and then reacts with the electron acceptor remaining in the medium which has the highest oxidation/reduction potential. This mechanism can be repeated until either the electron acceptors are exhausted or the bioreactive organic matter is exhausted.
the bioreactive structural units react in parallel: there does not exist a sequential mechanism in which a bioreactive structural unit would react once another bioreactive structural unit has been completely decomposed. In fact, all the bioreactive structural units can react at the same time but with different kinetics defined by individual reaction rates (Ri).

The identification and the quantification of the initial bioreactive structural units (stage 2 of the method) makes it possible to determine the coefficients x, y, z, v and w of equations 1, 2, 3 and 7 of the reaction scheme. In order to completely define this reaction scheme, it is necessary to define the different individual reaction rates (Ri) for the sequential reactions.

### Determination of the Individual Reaction Rates (Ri) by Evaluation of the Relative Losses of the Bioreactive Structural Units

The losses of the different bioreactive structural units are determined both with regard to the analysis of the rock extracts and with regard to that of those obtained by gentle pyrolysis of the solid organic matter. These two analyses are carried out on samples taken at increasing depths. The organic matter deposited in the sediments is sampled as a function of the depth, generally between 0 and 1500 m, but samples can be taken down to depths of 2500 m. Thus, an organic matter series is recovered at increasing depths of burial. The analysis of these samples is the same as in stage 2 of the method according to the invention.

The decrease in the amounts of bioreactive structural units as a function of the depth thus corresponds to the losses associated with the bacterial activities. Thus, with regard to a series of organic matter samples taken at different depths, the losses for each of the bioreactive structural units between two given depths are determined. The sum of these losses constitutes the total loss of the starting organic matter at a given level of biological alteration. Thus, a calculation is made of the losses of structural units for each of the depth zones corresponding to a given biological reaction.

Knowing the relative losses of the different bioreactive structural units as a function of the progress of the biological reaction, the various individual reaction rates (Ri) are determined for the sequential reactions.

It is possible, for example, to use the method described in patent application EP 2 154 254. The bioreactive structural units are categorized by decreasing order of loss as a function of the depth. Coefficients are then assigned, the greatest value of which is assigned to the most sensitive bioreactive structural unit. These coefficients are then optimized by comparing the relative proportions obtained between the structural units with those measured with regard to the natural samples as a function of the depth. Thus, on summing these optimized coefficients and on standardizing them at 100%, individual reaction rates (Ri) are obtained for the sequential reactions.

### 4—Determination of the Volumes of Biogenic Gases Generated

Each equation (1, 2, 3 and 7) is applied to each bioreactive structural unit. Among these equations, equation 7 describes the whole of the process for formation of the biogenic gas (CH₄). Thus, knowing the degree of loss of each of these structural units, equation 7 is used to calculate, with regard to each of the bioreactive structural units, the amounts of methane generated. On summing these amounts of methane generated from each of the bibreactive structural units, the total amount of methane produced is calculated.

### EXAMPLE

A weight of 1000 kg of sediments which contains 4% of organic matter, i.e. 40 kg of organic matter, is considered. If this organic matter at the beginning of methanogenesis contains 10% of the bioreactive structural unit C₄₀H₈₀O₂ (molecular weight equal to 592 g), this corresponds to a weight of 4 kg of acids. If 10% of this bioreactive structural unit are decomposed, this corresponds to a loss of 400 grams, i.e. 0.675 mol. On applying equation 7, the microbial decomposition of 0.675 mol of C₄₀H₈₀O₂ corresponds to the production of 19.9 mol or 446 liters of CH₄ per 1 ton of sediment.

1. Method for determining an amount of biogenic gas generated by biodegradation of organic matter in a sedimentary series of a sedimentary basin, characterized in that the following stages are carried out:
i. bioreactive structural units having formulae CₓH_{y}OᵥS_{z}N_{w} with x>6, y>x, v<6, z<6 and w<6 are considered;
ii. a sample of a rock of the sedimentary series is taken at a depth close to the surface, such that said rock has not been subjected to any biodegradation, and amounts of each bioreactive structural unit present in said sample are determined;
iii. a reaction scheme is defined in which said bioreactive structural units react in parallel with electron acceptors, and each of the bioreactive structural units reacts sequentially with said electron acceptors according to individual reaction rates for each bioreactive structural unit, said rates being determined by experimental measurements;
iv. the amount of biogenic gas produced by biodegradation of said bioreactive structural units is determined by means of said reaction scheme and said amounts of each structural unit present in said sample.

2. Method according to claim 1, in which the individual reaction rates are determined by carrying out the following stages:
a. rock samples are taken from a sedimentary series of a sedimentary basin, at the surface and at different depths;
b. a proportion by weight of each bioreactive structural unit is determined in each of said samples by means of extraction by an organic solvent, followed by pyrolysis;
c. an amount of biogenic gas produced by each bioreactive structural unit is determined by means of said reaction scheme and the proportions by weight;
d. the individual reaction rates of each bioreactive structural unit are deduced therefrom.

3. Method according to claim 1, in which said bioreactive structural units are grouped together into bioreactive structural groups.

4. Method according to claim 1, in which said biogenic gas is methane.

5. Method according to claim 1, in which said electron acceptors are chosen from the following acceptors: O₂, NO₃, SO₄ and H₂O.
